(19) United States
(12) Patent Application Publication
(10) Pub. No.: US 2010/0256733 A1
(43) Pub. Date: Oct. 7, 2010

(54) IMPLANT, METHOD AND DEVICE FOR PRODUCING AN IMPLANT OF THIS TYPE

(75) Inventor: Kirsi Schuessler, Pfinztal (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: ACANDIS GMBH & CO. KG, Pfinztal (DE)

(21) Appl. No.: 12/593,940

(22) PCT Filed: Mar. 28, 2009
(86) PCT No.: PCT/EP08/02509

§ 371 Date: Apr. 1, 2010

(30) Foreign Application Priority Data
Mar. 30, 2007 (DE) 10 2007 015 462.5

(51) Int. Cl. A61F 2/82

(52) U.S. Cl. 623/1.15

(57) ABSTRACT

An implant includes a wall element (10) which, when implanted, comes into contact with a fluid, the wall element (10) being adapted to influence the flow behaviour of the fluid. The wall element (10) has a non-continuous profile.

### CROSS-REFERENCE TO RELATED APPLICATIONS

This application is a Section 371 of International Application No. PCT/EP2008/002509, filed Mar. 28, 2008, which was published in the German language on Oct. 9, 2008, under International Publication No. WO 2008/119520 A1 and the disclosure of which is incorporated herein by reference.

### BACKGROUND OF THE INVENTION

The present invention relates to an implant, a method and a device for producing an implant of this type.

An implant, such as that described in EP 1 153 581 A1, is known. The narrowing of vessel sections (restenosis) following a stent implant continues to be a major clinical problem in spite of numerous further developments in this field. In this context, it is generally acknowledged that neointimal hyperplasia is the chief cause of restenosing following a stent implant. Although the mechanisms responsible for the occurrence of restenosis have not yet been fully clarified, the damage to the endothelium and the smooth muscle cells of the vessel wall, which occurs in the course of a stent implant, the reduced fit (compliance) due to the rigid stent structures and the change in shear stress at the vessel wall (wall shear stress) of the stented vessel sections are regarded as triggers of neointimal hyperplasia and, therefore, of restenosis.

Moreover, it is known that stented vessel sections not only lead to a change in the shear stress at the vessel wall, but also can promote the undesirable formation of thrombi due to changes in the haemodynamics of the natural blood flow. It is furthermore known that the formation of restenoses may depend on the stent geometry. It is generally assumed that a low shear stress at the vessel wall promotes neointimal hyperplasia.

According to the abovementioned EP 1 153 581 B1, a stent in the inside of which a flow cylinder is arranged centrally in the longitudinal direction of the stent is proposed in this connection. Due to the reduced flow diameter between the flow cylinder and the surrounding wall of the stent, the flow rate in the stent is increased, so that a steeper flow profile is established in the vicinity of the wall. Needless to say, fixing of the flow cylinder in the stent presents production problems. In addition, the flow cylinder impairs the flexibility of the stent.

WO 03/1057328 discloses a braided multilayer stent which has a core in the inside to influence the haemodynamics. There are also production problems with this stent.

FIG. 1 shows a conventional tubular stent arranged on a cylindrical mandrel as a shaping tool. In this conventional stent the design elements, for example the struts, lie on a cylindrical generated surface and form a wall element 110 with an inner surface 110a and an outer surface 110b. The inner surface 110a forms or limits a flow channel through which, when implanted, blood flows. The outer surface 110b lies against the vessel wall when implanted and supports this. As shown in FIG. 1, the inner surface 110a forms a flat, non-profiled generated surface. This means that all the design elements (struts, connectors, end curves) are arranged in the same plane, i.e. in the generated surface formed by the inner surface 110a.

The present invention is based on the object of providing an implant with a wall element which is comparatively easy to produce and is improved with respect to a reduction in restenosis following an implant. The object of the present invention is furthermore to provide a method and a device for producing an implant of this type.

### BRIEF SUMMARY OF THE INVENTION

According to the present invention, this object is achieved with respect to the implant, the method and the device described in the appending claims.

The present invention is accordingly based on the idea of providing an implant with a wall element which when implanted comes into contact with a fluid, the wall element being adapted to influence the flow behaviour of the fluid. According to the present invention, the wall element has a non-continuous profile.

The present invention pursues a different approach to that in the case of stents which have the effect of a reduction in the effective flow diameter due to a cylinder arranged in the flow channel. In the implant according to the present invention, the wall or a wall element is modified, and in particular by a non-continuous profiling thereof which influences the flow behaviour. This has the advantage that no additional flow body, such as the flow cylinder, has to be connected to the wall in an elaborate manner. Rather, a narrowing in cross-section is achieved by the wall element itself, and in particular by non-continuous profiling thereof.

The present invention has the advantage, in particular, that by the influencing of the haemodynamics in the blood stream, in particular by the increase in the flow rate, the shear stress at the vessel wall is increased and neointimal hyperplasia and restenosis are thus inhibited. Early endothelialization of the implant is furthermore promoted, which likewise has a positive effect since the occurrence of neointimal hyperplasia is thereby counteracted. The influencing of the flow dynamics, in particular the increase in flow rate, moreover inhibits the formation of thrombi.

A further advantage of the present invention is that the implant is particularly suitable for treatment of aneurysms. The narrow mesh width of aneurysm stents required for covering the aneurysm leads to adverse side effects, such as restenosis, and to the formation of thrombi. These undesirable side effects are avoided or at least occur to a lesser extent by the non-continuous profiling of the wall element.

The present invention is not limited to stents, in particular intraluminal stents, but includes implants generally, in particular medical or endovascular implants with an incident flow of fluid, or which generally have a flow conduction function, such as stent grafts, protection filters, etc.

In a preferred embodiment of the present invention, the wall element has profile elements with incident flow surfaces arranged non-continuously. The incident flow surfaces are not connected continuously to one another, but have generally abrupt transitions, so that overall a non-continuous profile or a non-continuous contouring of the inside of the wall element results. A wall element of this type or an implant with a wall element of this type can be produced easily and with comparatively low costs.

A particularly effective influencing of the flow behaviour is achieved if the incident flow surfaces have a main orientation which essentially extends in the direction of flow.

The incident flow surfaces can in each case be arranged, with respect to the wall element, with a tilt and/or a convex curve and/or a concave curve. By the different formation of the incident flow surfaces, a different influencing of the flow behaviour can be established.

In a preferred embodiment of the present invention, the non-continuous profiling of the wall element is achieved by the incident flow surfaces each having an end at a distance from the wall element, in particular a free end, which when implanted projects into the fluid flow. The profile elements here can form a wall element profile which is sawtooth-like in cross-section. A profile cross-section of this type can be produced in a simple manner.

The profile elements can form a pattern, in particular a helical pattern. As a result of the formation of a pattern, a desired flow behaviour is imprinted on the fluid over a longer flow distance.

The wall element can include a grid structure with grid elements, such as struts, end curves, connectors and the like, with at least some of the grid elements for formation of the incident flow surfaces each being arranged in a different plane to an inner surface of the wall element. This embodiment of the present invention is particularly suitable for stents and is distinguished by a production-friendly construction. No additional separate elements which are connected to the stent to form a wall profile are required for this. Rather, the non-continuous profile is formed by the grid elements already present, which are modified such that they are each arranged in a different plane to the inner surface of the wall element. The inner surface of the wall element is, therefore, not continuously flat, but has generally a contour of non-continuous profile.

The process according to the present invention for producing an implant of this type is based on the wall element having a non-continuous profile for influencing the flow behaviour of a fluid which comes into contact with the wall element when implanted. For this, the wall element can be formed locally such that non-continuously arranged incident flow surfaces of the profile elements are formed.

For production of an implant of this type, a device which comprises a moulding with a contour of non-continuous profile is proposed according to the present invention.

The present invention is explained in the following with further details with the aid of embodiment examples with reference to the attached drawings in diagram form.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a cross-sectional elevation view of a conventional implant without profiling, which is arranged on a shaping tool;

FIG. 2 is a cross-sectional elevation view of an implant with non-continuous profiling, which is arranged on a modified shaping tool, in accordance with a preferred embodiment of the present invention;

FIG. 3 is a schematic cross-sectional elevation view of an implant, when implanted, in accordance with another preferred embodiment of the present invention; and

FIG. 4 is a plan view of the implant shown in FIG. 3.

### DETAILED DESCRIPTION OF THE INVENTION

Certain terminology is used in the following description for convenience only and is not limiting. The words “inner,” “outer,” “inwardly” and “outwardly” refer to directions toward and away from, respectively, the geometric center of the implant or device and designated parts thereof. Additionally, the terms “a,” “an” and “the,” as used in the specification, mean “at least one.” The terminology includes the words above specifically mentioned, derivatives thereof, and words of similar import.

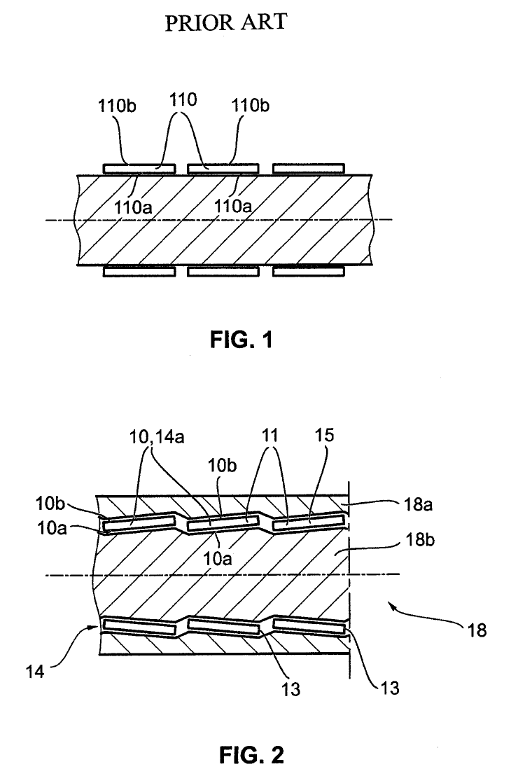

Referring to the drawings in detail, wherein like numerals indicate like elements throughout, there is shown in FIGS. 2-4 preferred embodiments of an implant. In contrast to the conventional implant shown in FIG. 1, the design elements or grid elements 14a of the implant according to a preferred embodiment of the present invention (FIG. 2) are arranged in a different plane to an inner surface 10a of non-profiled wall sections, and in particular such that a wall element 10 has a non-continuous profile. The grid elements 14a of the present embodiment form profile elements 11 with incident flow surfaces 12 or deflection surfaces for deflection of the fluid flow when implanted.

While the inner surface 110a of the stent according to FIG. 1 extends parallel to the longitudinal axis of the stent, the inner surface 10a of the implant according to FIG. 2-4 has sections which are not arranged parallel to the longitudinal axis. In particular, the inner surface 10a is tilted or angled in sections. This is achieved by at least some of the grid elements 14a being moved out and projecting out of the original generated surface of the implant. This results in the non-continuous profiling of the wall element 10, at least some grid elements 14a forming the original non-profiled generated surface of the wall element 10 in sections and some grid elements 14a projecting out of the generated surface with non-profiled sections. The grid elements 14a preferably project here only to a distance such that a flow channel, although formed with a reduced flow diameter, is opened.

The particular inner surface 10a of the projecting grid elements 14a functions here as an incident flow or deflection surface 12 for the fluid flow. The incident flow surfaces 12 of the various grid elements 14a and of the profile elements 11 are preferably arranged non-continuously and are interrupted at least in the profile space, i.e. outside the non-profiled original generated surface. This results generally in an abrupt or non-continuous transition between the profile elements 11.

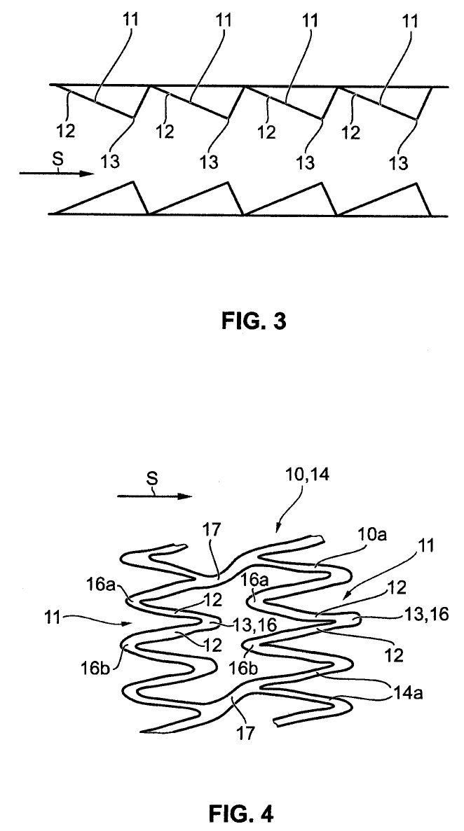

FIG. 3 shows the non-continuous form of the wall profile, the profile elements 11 having tips or ends 13 which project into the flow channel. As shown in FIG. 4, the ends 13 correspond to end curves 16, each of which connects the struts 15 arranged adjacent. When implanted, the ends 13 or the end curves 16 are preferably arranged radially inwardly at a distance from the wall sections which are non-profiled in sections or from the wall sections which chiefly fulfil a support function when implanted. In addition to the grid elements 14a projecting radially inwardly, it is also possible to provide grid elements 14a projecting radially outwardly, through which an improved anchoring of the stent in the surrounding tissue is achieved.

The incident flow surfaces 12 have a main or preferred orientation which essentially extends in the direction of flow, as shown by the arrow S in FIG. 3. This means that the incident flow surfaces 12 are angled in the same direction and therefore all have essentially the same angle of inclination. In this context, the incident flow surfaces 12, or the associated grid elements 14a, can be tilted, the incident flow surfaces 12 themselves being straight or flat in structure, as shown in FIG. 3. Alternatively or in addition, the incident flow surfaces 12, or the associated grid elements 14a, can have a concave or convex curve. Various angles of inclination for individual incident flow surfaces 12 or various radii of curvature can moreover be established. The grid elements 14a are tilted or curved uni-directionally, and in particular in the direction of flow. Various geometric properties can be combined with one another, in particular combined in the form of a pattern.

The grid structure 14 of the stent according to one preferred embodiment of the present invention is shown in FIG. 4. As shown in FIG. 2, the grid structure 14 may include the struts 15, at least some of the struts 15 or generally some of the grid elements 14a each being arranged in a different plane to an inner surface 10a of the wall element 10 in a non-profiled region to form the incident flow surfaces 12. For formation of the non-continuous profiling, at least two struts 12 arranged successively in the direction of flow S are curved radially inwardly or project radially inwardly with their end curves 16 or ends 13 or generally grid elements 14a. This means that when implanted, the struts 12 or end curves 16 curved radially inwardly project into the flow channel of the stent. The end curves 16a, 16b at right angles to the direction of flow S, i.e. bordering or adjacent in the peripheral direction, are in the original cylindrical generated surface of the stent and form a non-profiled region of the wall element 10. This means that the adjacent end curves 16a, 16b are arranged in the plane of the diagram and the end curves 16 arranged in between are arranged outside the plane of the diagram of FIG. 4. A non-continuous profiling of the wall element 10 is achieved in this manner, the non-profiled regions and the non-continuously profiled regions being arranged in different planes. The non-profiled regions are determined by the grid elements 14a, in particular the end curves 16a, 16b, arranged in the original generated surface. The non-continuously profiled regions are formed by the bent or projecting grid elements 14a, in particular the struts 12 or end curves 16a, 16b.

In other words, the inner surfaces 10a of the profiled and non-profiled regions of the wall elements 10 form intersecting planes. In the present example, the profile elements 11 are part of the wall element 10 and differ from the wall element 10 by the inwards projecting or exposed position or arrangement.

The forming, i.e. the bending, of the grid elements 14a can take place in the region between two end curves 16, 16a bordering a strut 15. The strut 15 being bent in the vicinity of one of the two end curves or, for example, in the middle of the strut, depending on how large the incident flow surface 12 thereby achieved is to be.

This principle can be applied to the entire stent, it being possible to achieve various patterns, for example a helical or spiral pattern, by appropriate arrangement of the profiling, i.e. the bent elements. In this context, the profiling extends over a certain length or part length of the stent.

The hollow cylindrical overall shape of the stent is retained.

Overall, the profile elements 11 form local and non-continuously arranged constrictions of the flow diameter of the implant when implanted.

To produce the implant or the stent according to FIGS. 2-4, the wall element 10 is preferably moved out of at least regions of the surface originally formed by the wall element 10. This can be effected, for example, by the struts 15 being curved radially inwardly and/or radially outwardly. As a result, the individual struts 15 are angled with respect to the original cylindrical generated surface of the stent. This is expediently effected by a shaping tool 18 shown in FIG. 2, which has a correspondingly profiled mandrel. The shaping tool 18 shown in FIG. 2 is divided into at least two and has an inner part 18b and an outer part 18a. The outer profile of the inner part 18b and the inner profile of the outer part 18a are complementary in structure, so that in the assembled state the shaping tool has a shape corresponding to the angled profile of the implant.

The wall element 10 or the implant can preferably be produced from a shape memory material, such as nitinol. This has the advantage that when the implant is compressed the wall element 10 can be forced into the same area as the other grid elements 14a which, when expanded, are responsible for the non-continuous profiling of the wall element. The implant can, therefore, be constructed without an additional increase in the size of the insertion diameter. By using a shape memory material for the wall element 10, the three-dimensional structure (in comparison with the two-dimensional structure of the non-profiled wall element) produced by the wall element when expanded is reduced to the previous two-dimensional form on insertion of the stent.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1-11. (canceled)

12. An implant comprising a wall element (10) which, when implanted, comes into contact with a fluid, the wall element (10) being adapted to influence the flow behaviour of the fluid, the wall element (10) having a non-continuous profile.

13. The implant according to claim 12 wherein the wall element (10) comprises profile elements (11) with non-continuously arranged incident flow surfaces (12).

14. The implant according to claim 13 wherein the incident flow surfaces (12) have a main orientation which essentially extends in the direction of flow.

15. The implant according to claim 14 wherein the incident flow surfaces (12) are each arranged, with respect to the wall element (10), with a tilt or a convex curve or a concave curve.

16. The implant according to claim 15 wherein the incident flow surfaces (12) each have a free end (13) at a distance from the wall element (10) projecting into the fluid flow when implanted.

17. The implant according to claim 13 wherein the profile elements (11) form a sawtooth profile of the wall element (10) in cross-section.

18. The implant according to claim 17 wherein the wall element (10) comprises a grid structure (14) with grid elements (14a), wherein at least some of the grid elements (14a) are each arranged in a different plane to an inner surface of the wall element (10) to form the incident flow surfaces (12).

19. The implant according to claim 18 wherein the profile elements (11) form a helical pattern.

20. A method for producing an implant comprising a wall element (10), wherein the wall element (10) has a non-continuous profile to influence the flow behaviour of a fluid which comes into contact with the wall element (10) when implanted.

21. The method according to claim 20 wherein the wall element (10) is formed locally to form profile elements (11) with non-continuously arranged incident flow surfaces (12).

22. A device for producing an implant comprising a wall element (10) with a moulding which has a contour with a non-continuous profile.
